(19) United States
(12) Patent Application Publication
(10) Pub. No.: US 2025/0339957 A1
(43) Pub. Date: Nov. 6, 2025

(54) LOW ENERGY, NON-FERROUS, NON-MAGNETIC DRIVING SYSTEM FOR MEDICAL AND OTHER APPLICATIONS

(71) Applicant: University of Iowa Research Foundation, Iowa City, IA (US)

(72) Inventors: Sandeep Laroia, Iowa City, IA (US); Venanzio Cichella, Iowa City, IA (US); Caterina Lamuta, Iowa City, IA (US)

(73) Assignee: University of Iowa Research Foundation, Iowa City, IA (US)

(21) Appl. No.: 18/870,291

(22) PCT Filed: Jul. 26, 2023
(86) PCT No.: PCT/US23/71040

§ 371 Date: Nov. 27, 2024

Related U.S. Application Data
(60) Provisional application No. 63/392,433, filed on Jul. 26, 2022.

(51) Int. Cl. B25J 9/10; A61B 5/055; A61B 6/03; A61B 34/00

(52) U.S. Cl. CPC B25J 9/104; A61B 5/055; A61B 6/032; A61B 34/70

(57) ABSTRACT

An actuator includes a housing formed from a non-ferrous, non-magnetic material, the housing having a front end and an opposite back end and a sidewall extending between the front end and the opposite back end, a member positioned within the housing, a first plurality of fibers operatively connected to the member. The actuator further includes a second plurality of fibers operatively connected to the member such that the second plurality of member are configured to allow for providing forces opposite of the first plurality of fibers and switching electrically connected to each of the first plurality of fibers and each of the second plurality of fibers to provide for selectively activating each of the first plurality of fibers and each of the second plurality of fibers. The housing and the fibers are non-magnetic and non-ferrous.

### RELATED APPLICATIONS

This application claims priority to U.S. Provisional Patent Application No. 63/392,433, filed Jul. 26, 2022, hereby incorporated by reference in its entirety.

### FIELD OF THE INVENTION

The present invention relates to actuators. More particularly, but not exclusively, the present invention relates to low energy, non-ferrous, non-magnetic actuators and a configuration of twisted and coiled artificial muscles forming such actuators.

### BACKGROUND OF THE ART

Although the background of the invention is discussed with emphasis on medical application, the present invention is not to be limited to this application.

Use of robots in health care is recognized as a potential solution to a variety of different problems. One potential benefit of the use of robots is to reduce costs associated with medical procedures. Given a current and impending shortage of skilled medical professionals, costs may be increased as this represents about 20 percent of total health costs. In addition to costs, access to appropriate in the United States is limited. Indeed, the U.S. ranks last on the developed nation list for access. This is especially true in underserved rural and inner-city areas. Robots are also seen as being able to potentially reduce patient complications such as bleeding or otherwise provide improved outcomes. Moreover, in some application robots allow for improved safety. For example, where ionized radiation doses are administered to a patient, where a robot insertion is used, the physician receives a dose of 0 μSv whereas if there is manual insertion there is a dose of 5.7 μSv on average (P<0.001). Personnel safety is also a concern in situations such as presented by the Covid-19 pandemic or the presence of other infectious diseases.

Yet, despite the general acknowledgement that robotics holds great promise in medical applications, numerous problems remain. For example, in the medical field, automated and semi-automated medical and other devices need actuators. Various actuation technologies with electromagnetic motors, electric motors and pneumatic devices may be used to drive robotics, prosthetic devices, or in other types of applications The energy outputs with these approaches are very impressive (e.g., ˜10 kW kg-1 for jet engines) however, existing electrical and electromagnetic actuators are bulky and rigid especially from a biomedical application point of view.

Moreover, there is a need of soft actuation technologies to, for example, mimic the functionalities of natural muscles. Recently artificial muscle technology has been described and in laboratory conditions, artificial muscles have been able to surpass the performance of their natural counterparts in some particular properties. For instance, dielectric elastomer actuators (DEA) are capable of producing strains of >300%; similarly thermal responsive coiled polymer fibers can achieve an impressive specific power of 27.1 kW kg-1 which is several times more than natural muscles.

Many medical and non-medical applications require motorized system for generation of adequate force for moving various parts of the equipment, however almost all of these have either ferrous or magnetic components, which may at times limit their use in specific situations. For example, metallic components of the existing motors would cause artifacts in the machines using x rays. Similarly ferrous containing machines cannot be used in magnetic fields of an MRI machine. This imposes limitations on developing more sophisticated medical devices using current ferrous electromagnetic technologies. Also, the existing metal-based technology can be miniaturized only to a certain extent.

What is needed are new technologies such as may be used to provide a novel light weight, non-ferrous, non-magnetic driving system/motor with low energy requirements, biocompatible and minimal moving parts which is suitable to be miniaturized.

### SUMMARY

Therefore, it is a primary object, feature, or advantage to improve over the state of the art.

It is a further object, feature, or advantage to provide actuators suitable for use in medical applications.

Another object, feature, or advantage is to provide actuators suitable for use in autonomous and semi-autonomous applications.

It is a still further object, feature, or advantage to provide actuators which may be used in robots which allow for inline work such that imaging and intervention can happen in real-time and simultaneously.

Another object, feature, or advantage is to provide actuators which enable robotic systems to be lightweight and not bulky.

Yet another object, feature, or advantage is to provide actuators which are efficient in operation with low energy requirements.

A still further object, feature, or advantage is to provide systems which are easy to set up for medical applications.

Another object, feature, or advantage is to provide systems which are compatible with existing workflows.

Yet another object, feature, or advantage is to provide systems which are compatible with existing imaging modalities such as CT scans.

A still further object, feature, or advantage is to provide systems which may be used inside a CT gantry for real-time control of procedures.

A further object, feature, or advantage is to provide systems with minimal space requirements.

Yet a further object, feature, or advantage is to provide systems which are portable and easily assembled.

A further object, feature, or advantage is to provide systems which allow existing systems such as existing CT scanners or MRI scanners to be retrofitted for autonomous or semi-autonomous operation.

A still further object, feature, or advantage is to provide systems which are safe for patients and health care providers.

Another object, feature or advantage is to provide robotic systems which can be operated by generalists who need not have the level of special training needed to perform procedures manually.

Another object, feature, or advantage is to provide an innovative lightweight, nonferrous, non-magnetic actuator system.

Yet another object, feature, or advantage is to provide an actuator system which is fully compatible with current diagnostic and scanning modalities.

A further object, feature, or advantage is to provide an actuator system with a minimal number of moving parts.

A still further object, feature, or advance is to use an actuator system which may use twisted and coiled artificial muscle (TCAM) technology.

Another object, feature, or advantage is to provide an actuator which incorporates opposing sets of TCAM fibers in order to address unidirectional TCAM contraction.

Yet another object, feature, or advantage is to provide actuators configured to translate linear movement to movement along curved paths.

A further object, feature, or advantage is to provide soft touch actuation.

One or more of these and/or other objects, features, or advantages will become apparent from the specification and claims that follow. No single embodiment need exhibit each and every object, feature, or advantage as different embodiments may have different objects, features, or advantages. Therefore, the claimed invention is not to be limited by or to these objects, features, or advantages.

According to one aspect, an actuator includes a housing formed from a non-ferrous, non-magnetic material, the housing having a front end and an opposite back end and a sidewall extending between the front end and the opposite back end. The actuator further includes a member such as a disc or shaft positioned within the housing, a first plurality of fibers operatively connected to the member, and a second plurality of fibers operatively connected to the member such that the second plurality of member are configured to allow for providing forces opposite of the first plurality of fibers. The actuator further includes switching electrically/thermally or by some other suitable activating energy connected to each of the first plurality of fibers and each of the second plurality of fibers to provide for selectively activating each of the first plurality of fibers and each of the second plurality of fibers. The housing consists essentially of materials which are non-magnetic and non-ferrous. The plurality of fibers consists essentially of materials which are non-magnetic and non-ferrous. However, based on the requirements, e.g., for non-medical uses, various substances can be used for the construction of these fibers.

According to another aspect, a robotic system for performing medical procedures is provided. The robotic system includes a gantry unit for positioning an effector unit, an effector unit operatively connected to the gantry unit, at least one surgical tool associated with the effector unit, and an actuator operatively connected to the surgical tool. The actuator includes a member operatively connected to a first set of TCAM or electroactive fibers and a second set of TCAM or electroactive fibers which are configured to provide opposing forces to the member to impart movement to the member.

The disclosed herein can also be used for non-medical purposes, e.g., activating non-medical robots to be used in industry where magnetic and aqueous environment does not allow use of traditional ferromagnetic motors. Some potential uses can be underwater/space applications. Controlling solar panels or light control for greenhouses and homes without use of large amounts of electricity can be some other examples.

### BRIEF DESCRIPTION OF THE DRAWINGS

FIG. 1 illustrates one example of an actuator.

FIG. 2 is another view of the actuator with the housing not present.

FIG. 3 is another view of the actuator with the housing not present and in a different position than shown in FIG. 2.

FIG. 4 illustrates one example of a system which includes an imaging device in the form of a CT scanner, and which has an effector unit including the actuator.

FIG. 5 is another view of the effector unit containing the actuator and mounted on a gantry.

FIG. 6 is another view of the effector unit which includes the actuator.

FIG. 7 illustrates an effector unit with subunits.

FIG. 8 is a block diagram of a system including an effector unit containing the actuator.

FIG. 9 is another view of the effector unit containing the actuator.

FIG. 10 is another example of an actuator.

### DETAILED DESCRIPTION

The automated and semi automated medical and other devices need actuators. Various actuation technologies with electromagnetic motors, electric motors and pneumatic devices have been traditionally utilized to drive the robotics and prosthetic devices so far. The energy outputs with these approaches are very impressive (e.g., ˜10 kW kg-1 for jet engines) however, existing electrical and electromagnetic actuators are bulky and rigid especially from biomedical applications point of view. The development of soft actuation technologies mimicking the functionalities of natural muscles is needed. Recently artificial muscle technology has been described and in the laboratory conditions, artificial muscles have been able to surpass the performance of their natural counterparts in some particular properties. For instance, dielectric elastomer actuators (DEA) are capable of producing strains of >300%; similarly thermal responsive coiled polymer fibers can achieve an impressive specific power of 27.1 kW kg-1 which is several times more than natural muscles.

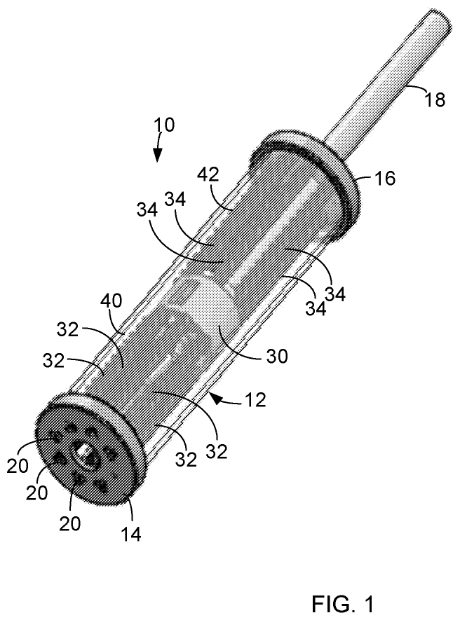

FIG. 1 illustrates one example of an embodiment of an actuator 10. As shown in FIG. 1, the actuator 10 has a housing 12. The housing 12 may be an outer casing which may be cylindrically shaped. The housing 12 has a first end 14 and an opposite/second end 16. A plurality of apertures 20 may be present on the first end 14. A plurality of apertures (not shown) may also be present on the second end 16. A disc 30 is shown which may move along the inner walls of the housing and may be centrally positioned. The disc 30 may be operatively connected in any number of different mechanical arrangements. For example, a rack and pinion or other type of tracking system may be used to allow the disc 30 to move along interior walls of the housing 12. The presence of the disc 30 may divide the interior of the housing 12 into a first chamber 40 and a second chamber 42. A shaft 18 is shown which may be centrally positioned in the housing 12.

The disc 30 may attach to a plurality of strands or fibers of electroactive polymer (EAP) or TCAM. In some embodiments, the plurality of strands or fibers may include different types of materials. A first set of fibers 32 are connected between the disc 30 and a front wall on the first end 14. A second set of fibers 34 are connected between the disc and a back wall on the second end 16.

Each of the fibers 32, 34 may be electrically connected to a switch or off and on circuit, so that each strand can be activated in a unique fashion based on the system requirements for a particular application.

For example, when all of the first set of fibers 32 are activated simultaneously and the fibers of the second set of fibers 34 are not activated, the disc 30 would move forward towards the first end 14. Similarly, if all of the second set of fibers 34 are activated but the first set of fibers 32 are not activated then the disc 30 would move backward towards the second end.

It should be understood that more precise control of the fibers 32, 34 may be used to apply any desired movement pattern. For example, by selectively activating specific fibers or groups of fibers the attached equipment may be made to tilt or rotate.

The central disc 30 is one example of a member. In other embodiments instead of a central disc 30, a suitably mounted central shaft may be used. The shaft may be used to hold different devices. Around the shaft, a suitable fiber may be wound in a spiral fashion. The spiral fiber in turn is attached to EAP/TCAM fibers attached to the side walls of the casing cylinder. When these horizontally placed EAP/TCAM fibers contract in a sequential manner, the motor shaft can be rotated clockwise or anticlockwise. A combination of the above-described motions allows a three-dimensional working environment for the motor. As EAP/TCAM strands can be made very small, so the motor may be adapted to fit into very constrained spaces also. In some embodiments, the member may be a combination of a central disc and a larger stabilizing disc. It is to be understood that the member may have different configurations. The central disc 30 may also have other shapes and geometries and may be considered a plate.

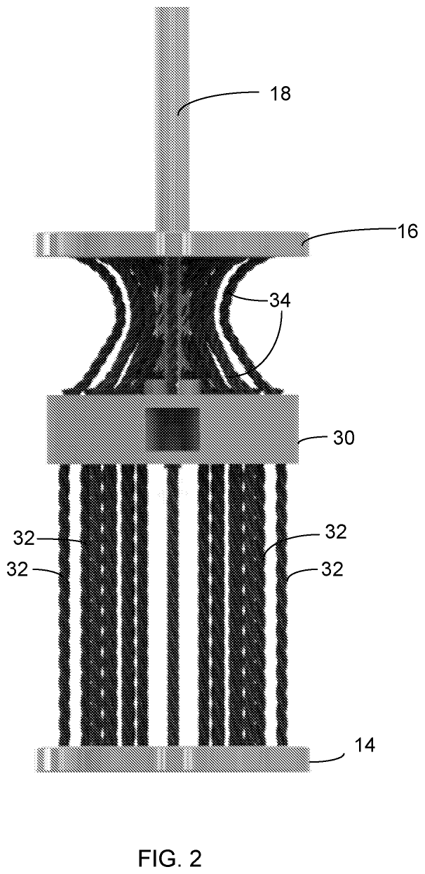

FIG. 2 is another view of an actuator with the housing not present. In FIG. 2, note the location of the disc 30 relative to the first end 14 and the second end 16. This position is achieved based on the ability to control the first set of fibers 32 and the second set of fibers 34. In FIG. 2, the second set of fibers or at least a portion thereof are at least partially activated thereby providing more force on the disc 30 then provided by any opposing force from the second set of fibers 34. Thus, the disc 30 is positioned closer to the second end 16 than the first end 14.

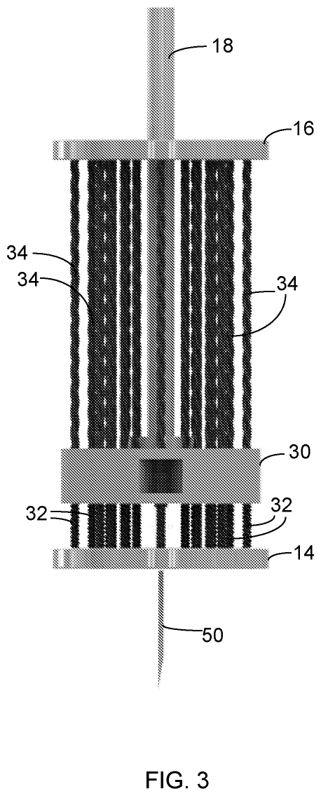

FIG. 3 is another view of the actuator with the housing not present. FIG. 3 is similar to FIG. 2, but the actuator is in a different position. In FIG. 3, note the location of the disc 30 relative to the first end 14 and the second end 16. This position is again achieved based on the ability to control the first set of fibers 32 and the second set of fibers 34 which are configured to provide opposing forces. In FIG. 3, the first set of fibers 32 is strongly activated resulting in the disc 30 being positioned very close to the first end 14. In addition, a device associated with the actuator, in this instance a needle 50 extends downwardly or outwardly from the first end 14.

Different geometric configurations are contemplated which allow for the fibers to be positioned to provide for movement in a manner which can control devices such as needles, syringes, scalpels, or other types of devices or tools. The actuators may also be differently configured. In addition, the actuator may provide linear and/or rotary movement depending upon its configuration.

According to one aspect, a surgical robot is provided. The surgical robot may be configured for performing one or more different types of procedures which may use devices such as scalpels, needles, or other types of devices. In one application, the surgical robot with the actuator may be used to provide for inline computed tomography (CT) scanner use for abscess drainage.

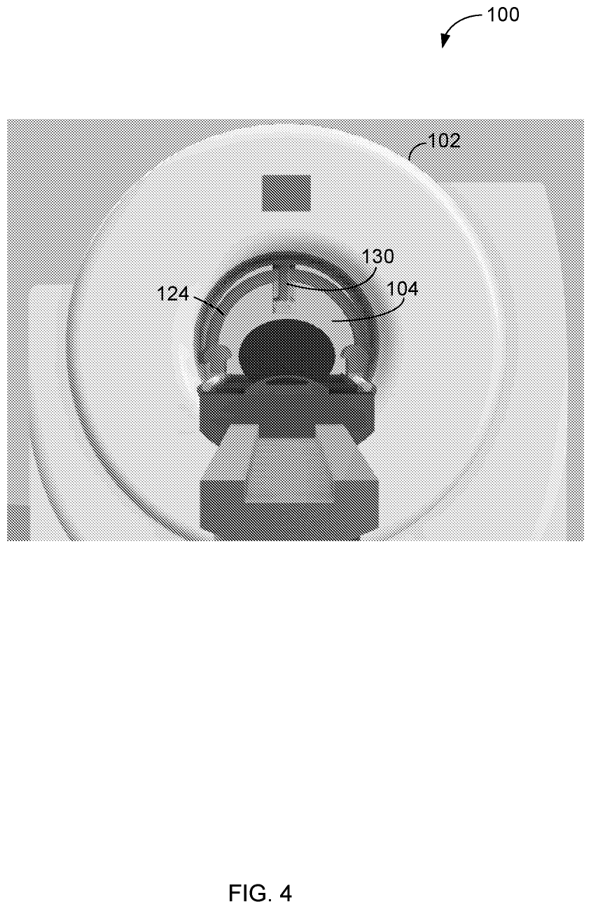

FIG. 4 illustrates one example of a system 100 which includes an imaging device in the form of a CT scanner 102 which has a chamber 104. A rail 124 of a gantry is positioned within the chamber 104. An effector unit 130 is operatively connected to the rail 124 of the gantry and may move along the rail 124 of the gantry and a second rail (not shown). Thus, the effector unit 130 may move in three dimensions relative to a patient in order to perform a procedure on the patient.

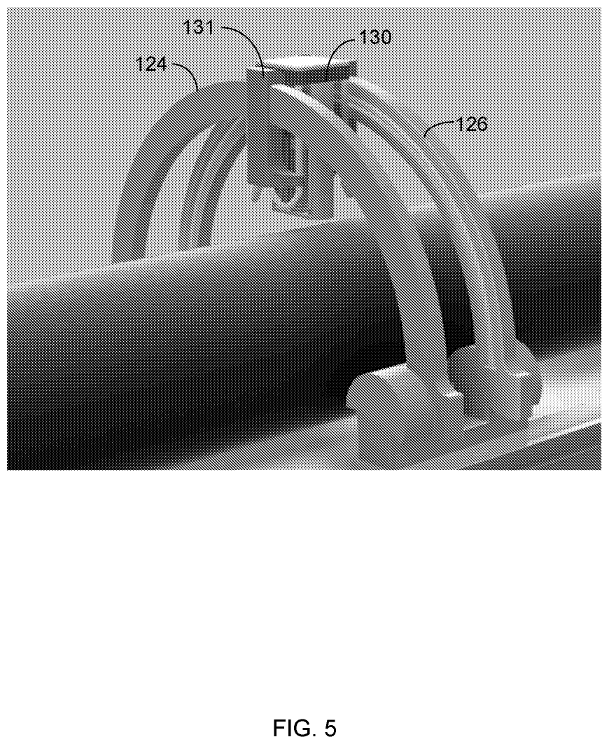

FIG. 5 is another view of the effector unit 130 operatively connected to rails 124, 126 of a gantry. The effector unit 130 may move along the rails 124, 126 and the rails may move relative to a patient so that precise positioning of the effector unit 130 relative to a patient may be performed. A TCAM-based motor 131 may provide for moving the effector unit 130 along the curved gantry. The use of the TCAMs allows for the elimination of more convention actuation systems, such as stepper motors and pneumatics. The TCAM-based motor is configured to allow for translation of linear movement to movement along a curved path of the rails of the gantry.

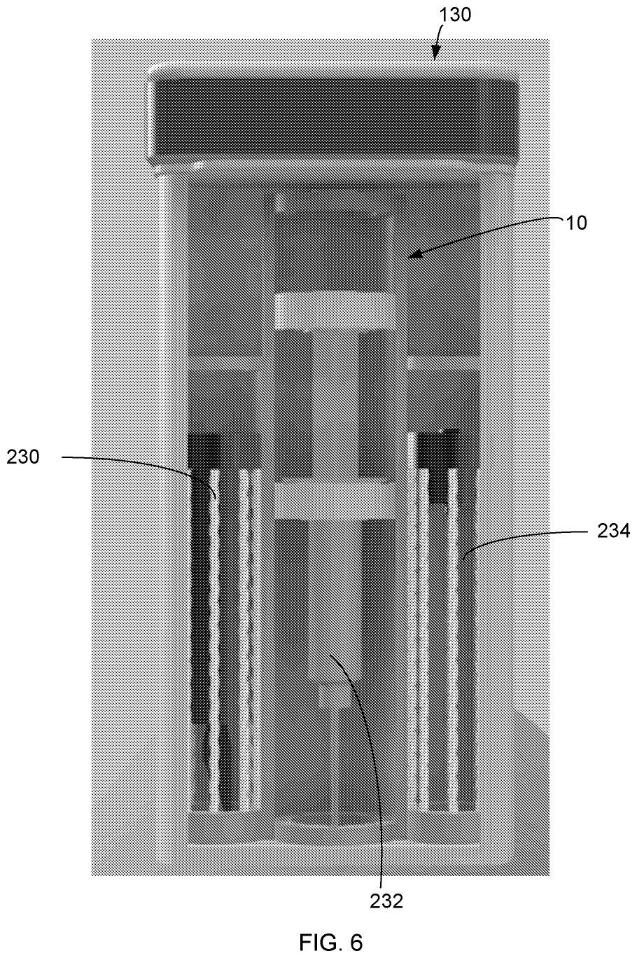

FIG. 6 illustrates another view of an embodiment of the actuator 10 within the effector unit 130. The effector unit 130 shown is configured with multiple surgical tools or subunits. A syringe subunit 232 is shown which may be activated by the TCAM fibers. A scalpel subunit 230 may be present. Cannula units or other suitable units may also be similarly activated as may be an aspiration needle subunit 234. In operation, the effector unit 130 provides the necessary tools to perform a medical procedure.

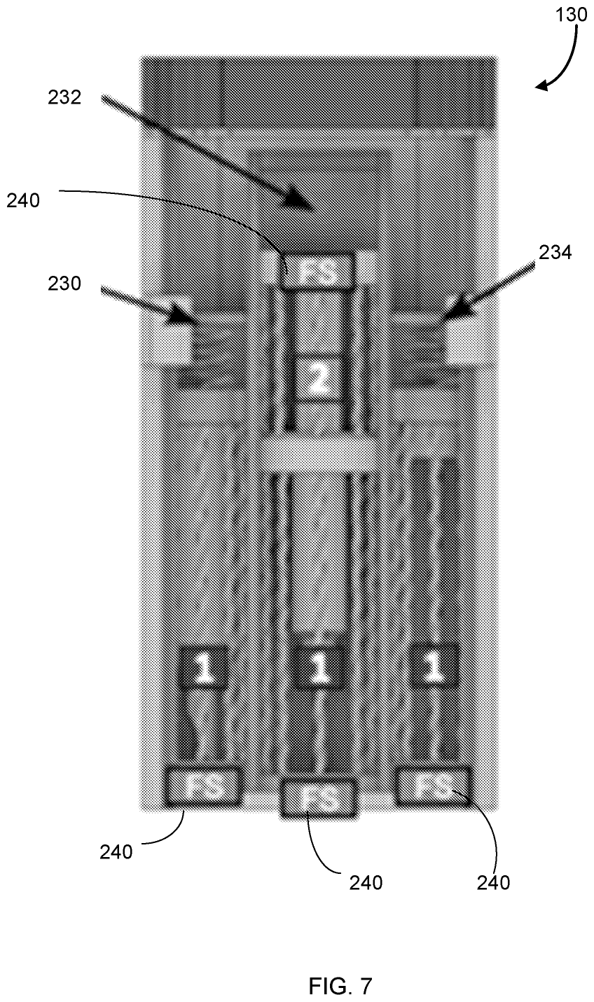

FIG. 7 illustrates another example of the effector unit 130. The effector unit 130 includes subunits including a scalpel subunit 230, a syringe subunit 232, and a needle or cannula subunit 234. Force sensors 240 may also be positioned at various locations throughout to measure force associated with the TCAM fibers. Note the configuration of TCAM fibers in the syringe subunit 232 allows for syringe plunger depression. Contraction of TCAM fibers also allows for downward vertical movement of each subunit.

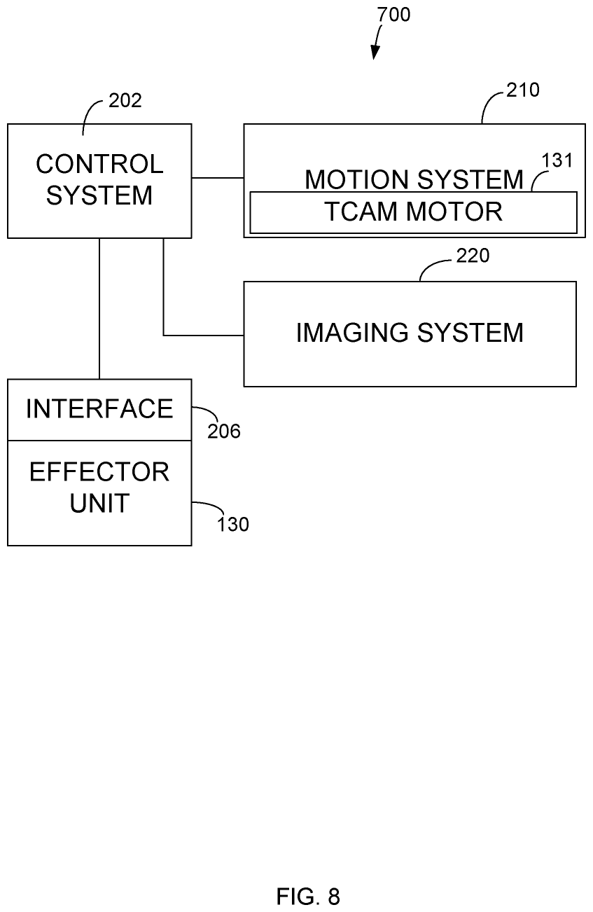

FIG. 8 illustrates one example of a system 700. The system includes a control system 202 which is operatively connected to a motion system 210 and an imaging system 220. The control system 202 is also operatively connected through an interface 206 to the effector unit 130, including one or more actuators within the effector unit and to strands of fibers of the actuators. Thus, the control system 202 provides for switching of the stands of fibers and may provide for individually activating a fiber or activating a set of fibers or all of the fibers. The control system 202 may be an electronic control system and may be programmed or otherwise configured to provide for control including determining which of the fibers to activate and when in order to provide the desired motion and/or behavior. The motion system 210 may be used for positioning the effector unit 130 and its tools relative to a patient. In some embodiments, the motion system 210 may include a TCAM motor 131 for moving the effector unit 130 along curved rails of a gantry unit. The imaging system 220 which may be a conventional CT-scanner or MRI scanner may be used to provide imaging information to assist with placement and operation of tools. It is contemplated that in some embodiments, a skilled health care provider may perform or supervise performance of a procedure remotely. In some embodiments, the procedure may be performed at least semi-autonomously. Where the robot functions semi-autonomously, image-guided AI and control algorithms may be used.

The interface 206 allows the control system 202 to communicate with the effector unit. The interface 206 may include analog and/or digital signals. For example, in some embodiments, the control system 202 may send voltage signals for activating different strands of an actuator through the interface 206.

The control system 202 may include one or more processors and/or microcontrollers. The control system 202 may be used to selectively activate TCAM fibers. The TCAMs may be actuated using an external battery.

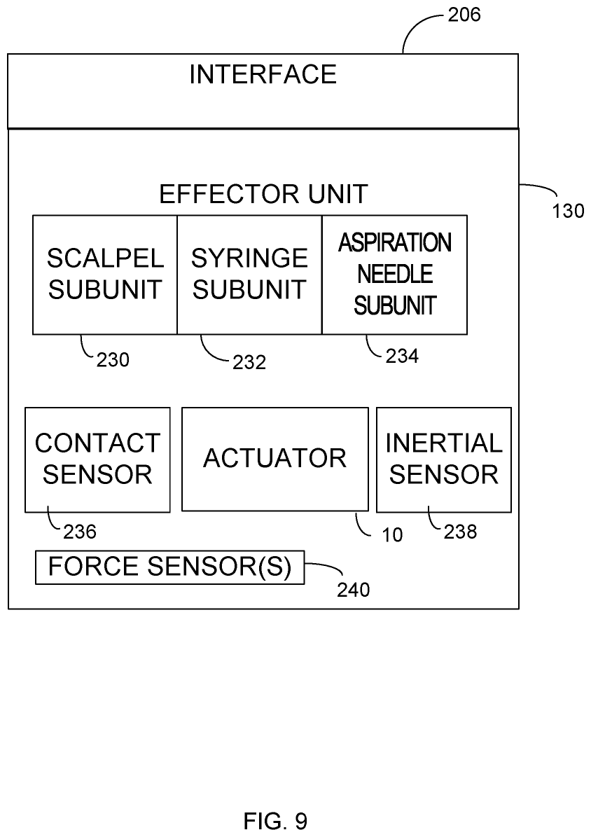

FIG. 9 is a block diagram illustrating the effector unit 130 with an interface 206. As shown, the effector unit 130 includes a scalpel subunit 230, a syringe subunit 232, and an aspiration needle subunit 234. The effector unit 130 also includes one or more actuators 10. In some embodiments, each subunit may have its own actuators. In other embodiments, some actuators may be used for more than one subunit. The effector unit 130 may further include one or more sensors such as a contact sensor 236. The contact sensor 236 may be used to determine that the effector unit 130 is in physical contact with a patient. One example of a contact sensor 236 which may be used is a capacitive sensor. An inertial sensor 238 may also be present in the effector unit 130. The inertial sensor 238 may be in the form of an inertial measurement unit (IMU) for positional control. One or more force sensors 240 may also be present. The force sensors 240 may be used within the effector unit 130 to measure the external force output by the TCAM fibers to ensure that the proper force is applied to the patient through each subunit. Any number of TCAM fibers may be connected in parallel and actuated in tandem using an external battery and control system.

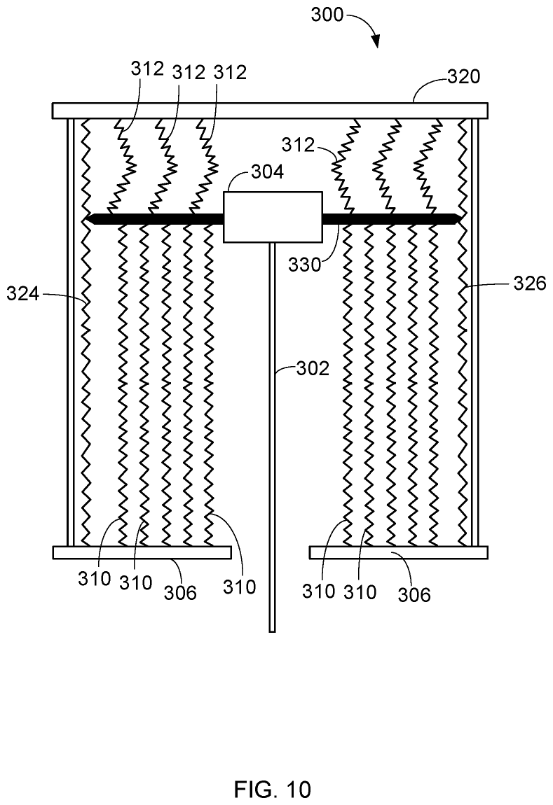

FIG. 10 illustrates another example of an actuator. As shown in FIG. 9, an actuator 300 has a first end 320 at the top and an opposite second end 306 at the bottom. A central member which may be in the form of a disc 304 is shown along within a thinner stabilizing disc 330 which may extend around the central disc 304. Gearings 324, 326 are shown which may be positioned along an inner wall of the housing (which may be a cylindrical tube) and the gearings 324, 326 may mesh with the outermost portions of the disc. The gearings 324, 326 may thus provide a rack and pinion configuration. There is a set of strands 310 operatively connected between the disc and the second end. There is another set of strands 312 operatively connected between the disc and the first end. By selectively activating these strands 310, 312, movement may be controlled. For example, movement of a rod 302 may be forward when strands 310 are activated. It should also be understood that movement may be linear so as to move the disc upward or downward within the tube. It should also be understood that movement may also be rotational to twist or turn.

The example of FIG. 10 may be beneficial in several respects. For example, a greater number of strands may be placed for force generation and the strands may be more densely packed. In addition, the strands 310 may be longer than the strands 312 when both are in an inactive state. The shorter strands 312 may be used to retrack. These strands may be folded in order to occupy less space. There also may be fewer strands 312 than there are strands 310. For example, in one embodiment, the actuator is not more than 12 cm in height and the movement of the piston is at least 6 cm and preferably at least 10 cm. The strands or fibers may be selected in type, size, and number to generate 20 Newtons of force or more or less depending on the application.

Thus, that the actuator shown and described is especially suited for medical applications as it can be used in environments where regular or conventional motors cannot be used because of weight, energy requirements or special circumstances like degradation of x-ray based imaging or dangerous displacements of ferrous components by the magnetic fields of magnetic resonance imaging (MRI) systems. Thus, the actuator is especially attractive for image guided procedures as the effector unit may be placed within the, for example, a CT machine aperture simultaneously with a patient.

It is to be further understood that the any number of different procedures may be performed using the actuator. One such an example of a procedure is where an inline CT scanner is used for abscess drainage. In draining an abscess different surgical tools may be used. This may include a syringe such as for administering an anesthetic to skin over the abscess. A scalpel may be used to form a small incision through the numbed skin. An aspiration needle or catheter may be inserted through the skin and into the abscess to remove or draw in the infected fluid. The inline CT scanner or other imaging system allows for monitoring of the process and may be especially helpful when the procedure is being supervised remotely and where there is automation. For example, image processing may be performed on acquired imagery which is used to generate appropriate commands for the control system to position the surgical tools relative to the abscess and to activate the tools in an appropriate manner. This may include identifying which strands of an actuator to activate, computing necessary force, and applying the necessary force, and other computations associated with the actuator.

As previously explained, the stands may be twisted and coiled artificial muscles (TCAMs). TCAMs may be formed from polymer fibers. TCAMs may be powered in various ways such as electrically, photonically, chemically, thermally, or otherwise. Thus, although it is recognized that instead of applying an electrical current to activate the strands of the actuator, alternative methods may be used. However, for purposes here, electrical activation is a convenient and efficient method of activation.

In the context of medical applications, a system may include a gantry unit or gantry arch with curved rails and which is positioned above a patient. Gantry rails enable the arch to traverse along a long axis of a bed. A TCAM based effector unit such as described houses the subunits necessary for anesthetization, incision creation, and aspiration of fluid, and a TCAM based motor may provide for moving the effector unit along the curved gantry. The use of the TCAMs allows for the elimination of more convention actuation systems, such as stepper motors and pneumatics. The effector unit may be mounted on the curved rails of the gantry.

The TCAM motor housing includes an outer cylindrical casing with holes cut in a circular pattern into the faces at either end of the cylinder. These holes may act as mounting points for the TCAMs, which extend from the end of the cylinder to a member which may be in the form of a central plate or disc in the middle. The extension/contraction of the opposing TCAM units enables controlled movement of the central plate or disc, which can then be used as an actuation input to other systems such as sub-units containing the needle/cannula, syringe, and scalpel.

Although various examples have been shown and described herein, the present invention contemplates numerous options, variations, and alternatives as may be appropriate in a particular application or environment.

It should further be understood that the technology shown and described herein has both medical applications and non-medical applications. Examples of non-medical applications include activating non-medical robots to be used in industry where magnetic and aqueous environments do not allow use of traditional ferromagnetic motors. Some potential uses can be underwater/space applications. Controlling solar panels or light control for greenhouses and homes without use of large amounts of electricity can be some other examples. Thus, the present invention contemplates a number of applications.

What is claimed:

1. An actuator comprising:
a housing formed from a non-ferrous, non-magnetic material, the housing having a front end and an opposite back end and a sidewall extending between the front end and the opposite back end;
a member positioned within the housing;
a first plurality of fibers operatively connected to the member;
a second plurality of fibers operatively connected to the member such that the second plurality of fibers are configured to allow for providing forces opposite of the first plurality of fibers; and
switching electrically connected to each of the first plurality of fibers and each of the second plurality of fibers to provide for selectively activating each of the first plurality of fibers and each of the second plurality of fibers;
wherein the housing consists essentially of materials which are non-magnetic and non-ferrous;
wherein each of the first plurality of fibers and the second plurality of fibers consist essentially of materials which are non-magnetic and non-ferrous.

2. The actuator of claim 1 wherein the first plurality of fibers forms a first twisted and coiled artificial muscle and the second plurality of fibers forms a second twisted and coiled artificial muscle.

3. The actuator of claim 2 wherein the housing is a cylindrically shaped casing.

4. The actuator of claim 3 wherein the member comprises a shaft.

5. The actuator of claim 3 wherein the member comprises a centrally positioned disc configured to move along inner walls of the housing.

6. The actuator of claim 5 wherein the centrally positioned disc is configured to move along the inner walls of the housing using a rack and pinion configuration.

7. The actuator of claim 3 wherein the member comprises a centrally positioned disc and a stabilizing disc.

8. The actuator of claim 1 wherein an electronic control system includes the switching for selectively activating each of the first plurality of fibers and selectively activating each of the second plurality of fibers.

9. The actuator of claim 8 wherein the electronic control system is configured to selectively activate specific fibers within the first plurality of fibers and the second plurality of fibers to provide at least one of tilt and rotation.

10. A system comprising the actuator of claim 9 and further comprising an imaging system operatively connected to the electronic control system and configured for acquiring imagery of a medical procedure associated with the actuator.

11. The system of claim 10 wherein the imaging system is selected from a set including a computed tomography (CT) scanner and a magnetic resonance imaging (MRI) scanner.

12. A robotic system for performing medical procedures, the robotic system comprising:
a gantry unit for positioning an effector unit;
an effector unit operatively connected to the gantry unit;
at least one surgical tool associated with the effector unit; and
an actuator operatively connected to the surgical tool;
wherein the actuator includes a member operatively connected to a first set of fibers of twisted and coiled artificial muscles (TCAM) and a second set of fibers of twisted and coiled artificial muscles which are configured relative to the first set of fibers of twisted and coiled artificial muscles to provide opposing forces to the member to impart movement to the member.

13. The robotic system of claim 12 wherein the actuator further includes a cylindrical housing.

14. The robotic system of claim 13 wherein the member comprises a disc within the cylindrical housing.

15. The robotic system of claim 14 wherein gearing is positioned along an interior wall of the cylindrical housing.

16. The robotic system of claim 12 further comprising a control system interfaced to the actuator for selective activation of the first set of fibers of TCAM and the second set of fibers of TCAM.

17. The robotic system of claim 16 further comprising an imaging system operatively connected to the control system.

18. The robotic system of claim 12 wherein the effector unit is mounted on curved rails of the gantry unit and wherein the effector unit comprises a TCAM based motor configured to move the effector unit along the curved rails of the gantry unit.

19. The robotic system of claim 12 wherein in an inactive state the first set of fibers of TCAM are longer than the second set of fibers of TCAM.

20. The robotic system of claim 12 wherein the at least one surgical tool includes at least one of a scalpel, a syringe, and an aspiration needle.
